(19) United States
(12) Patent Application Publication
(10) Pub. No.: US 2014/0155812 A1
(43) Pub. Date: Jun. 5, 2014

(54) Epilation

(71) Applicant: Yair Lauber, Givat Shmuel (IL)

(72) Inventor: Yair Lauber, Givat Shmuel (IL)

(73) Assignee: SCIENTIFIC-WIZARD LTD., Givat Shmuel (IL)

(21) Appl. No.: 14/233,898

(22) PCT Filed: Jul. 18, 2011
(86) PCT No.: PCT/IL11/00568

§ 371 Date: Jan. 21, 2014

Related U.S. Application Data
(60) Provisional application No. 61/344,447, filed on Jul. 26, 2010.

(51) Int. Cl. A61N 1/40; A61B 18/04; A45D 26/00

(52) U.S. Cl. CPC A61N 1/406; A45D 26/0014; A61B 18/04
USPC 604/20; 606/133; 604/506; 606/134; 606/27

(57) ABSTRACT

A method of removing at least one hair from a mammal comprising the steps of associating electromagnetic responsive species to the root of at least one hair and exposing the electromagnetic responsive species to an oscillating electromagnetic field.

### FIELD OF THE INVENTION

The present invention is directed to a method of removing hair, including body, facial, scalp and pubic hair.

### BACKGROUND OF THE INVENTION

Hair follicles are tube-like pockets of the epidermis that extend most or all the depth of the skin and enclose a small papilla of dermis at their base. The hair bulb, at the base of the hair follicle is a structure of actively growing cells, attached to which are one or more sebaceous glands. The follicle eventually produces a fine cylinder of hair. It will be appreciated that hair outside the follicle is dead.

Most mammals are covered with hair or fur. Human beings are somewhat exceptional in this regard, and are largely hairless, resulting in the species being dubbed the naked ape. Humans do, however, typically have hair on their heads. In addition, adult humans also typically have hair growth in the groin region and armpits, this adult hair growth, that occurs with the onset of puberty is known as pubic hair. Furthermore, adult men especially, have facial hair known as beards, and may have some body hair as well.

Different ethnic sub-species are more or less prone to body hair. Though indicating sexual maturity, excessive body hair it is sometimes seen as indicative of man's relationship to other animals. Whereas long, dense hair on the scalp is considered feminine and attractive, facial hair and heavy body hair on women is often indicative of hormone imbalances and is considered masculine and unsightly. Many women dye their hair, typically bleaching to a blond colour considered sexually attractive. Dark body hair coupled with blond scalp hair indicates that the scalp hair is a sham. Indeed, in many cultures, women find body hair unsightly or offensive and take pains to remove same. Whereas beards are considered manly in some cultures, in others, men also remove facial hair. Priests and others shave the scalp.

Balding is considered a masculine trait, but in some cultures is considered undesirable.

For surgical purposes, the removal of hair from a body part to be operated on may be required. In general, however, hair removal is performed for cosmetic reasons.

There are very many inventions and techniques directed to removing facial and body hair from humans. These techniques may be categorized as either depilation, which is the removal of hair at or near to the level of the skin, and epilation, which is the removal of hair below the skin, by removing from the hair follicle. Depilation lasts from hours to days. Epilation lasts for several days to weeks. Some methods of epilation are considered permanent. Although usually requiring repeating a number of times, permanent epilation weakens the hair in the follicle and eventually prevents recurring hair growth.

Depilation includes shaving with a razor or with an electrical shaver, either with rotary blades or blades behind a foil, and removal of hair with depilation creams which are chemicals, typically applied as a cream. Electrical shavers may result in rashes. Chemical depilatories work by breaking the disulfide bonds that link the protein chains that give hair its strength, making the hair disintegrate. Some people have religious convictions that make use of a razor unacceptable, and shaving simply cuts the hair near the base and, since new hair grows almost immediately, results in shadow or stubble. Indeed, most methods of depilation require repeated hair removal.

Epilation involves the removal of the hair at the root, and includes tweezing and plucking in which individual hairs are removed, and waxing and sugaring, wherein all hair within a defined area is removed.

Waxing involves spreading a warm wax over the area for epilation, allowing the wax to set, whereupon it hardens around the hair, and then removing in a swift action. The technique has the advantage that it removes both long and short hair, and that since it plucks the hair out of the follicle and thus results in a smooth finish, and repeated waxing weakens the follicle and eventually results in weaker hair growth that is more easily removed. It is, however, somewhat painful.

So called, permanent methods of epilation involve radiating the skin area with a laser, usually after first shaving. The laser treatment causes the hair root in the follicle to become damaged by heat. Repeated treatment results in hair growth being stunted. Other forms of radiation, such as exposure to X-rays will also result in hair loss by hair falling out, as is well known to chemotherapy patients. Usually however, the hair loss is temporary. Because of the risk of cell mutation, such techniques are not approved for cosmetic purposes.

Despite the plethora of techniques available, there is an ongoing need for more effective, less painful methods of depilation that preferably have increased times between repeated treatments, and most preferably prevent hair growth.

Head-shaving is a part of some Buddhist, Christian, Jain and Hindu traditions. Buddhist and Christian monks generally undergo some form of head-shaving or tonsure during their ordination; in Thailand monks shave their eyebrows as well. Brahmin children have their heads ritualistically shaved before beginning school.

In some parts of the Theravada Buddhist world, it is common practice to shave the heads of children. Weak or sickly children are often left with a small topknot of hair, to gauge their health and mark them for special treatment. When health improves, the lock is cut off.

In Judaism, men are prohibited from shaving their beards or sideburns using a razor blade directly against the skin. However, electric razors may be used, since the cutting blade does not directly touch the skin. There is an additional prohibition to remove sideburns may shorter than the bottom of the man's cheekbone based on the verse in Leviticus 19:27.

Men also may not remove armpit or pubic hair by any means, as this is considered to be something that women do (Shulchan Aruch Yoreh Deah 182). Jewish women may shave freely in Judaism, even using a blade or razor. In a few extreme Hasidic sects, married women traditionally shave their heads bald since they are prohibited to expose their hair to men other than their husbands and families. However, the vast majority of Orthodox Jews reject this practice as a form of self-mutilation. Furthermore, both men and women are forbidden by Jewish law to cut their hair and men are forbidden to shave during a 30-day mourning period after the death of an immediate family member.

In contrast the Bandá'í religion explicitly prohibits head-shaving. Sikhs take an even stronger stance, opposing all forms of hair removal.

Muslim law (Sharia) puts hair in three categories: that which it is recommended to remove (moustache, pubic and armpit hair), that which it is recommended to keep (the beard), and that which is the object of limited recommendation (foot, hand, back, and chest hair). Removal of armpit and pubic hair is a hygienic practice which was taught by Muhammad and which was enumerated as having been part of practices conforming to man's premortal (Fitrah) nature practiced by all Prophets of God. On the other hand, shaving or removing part of the eyebrows is forbidden, although stray hairs between the eyebrows may be removed. Additionally, Muslim males may trim or cut hair on their heads. Also, males may trim hair on the chest and back but may not remove it completely, as this is considered to be too feminine. In contrast, women are permitted to remove hair from these areas. Since the 9th century, Muslim women have used chemical depilatories.

Permanent hair removal is desirable in certain cases, such as for transgender males to females, where full beard removal is desirable.

The only really effective permanent hair removal is by exposure to highly penetrating radiation such as Xrays. Due to the dangers of such radiation, which can have major adverse affects on health, such treatments are illegal in many places, and certainly to be discouraged.

Of the available solutions, there are several imperfect options. A number of methods have been developed that use chemicals, energy of varying types, or a combination to target the areas that regulate hair growth. Permanently destroying these areas while sparing surrounding tissue remains a difficult challenge. Methods include: Electroepilation, Electrolysis or electrology, Thermolysis and blends or combinations of electrolysis and thermolysis, and Photoepilation, which includes Laser hair removal using lasers and laser diodes, and Intense pulsed light using high-energy lamps, Diode epilation (high energy LEDs but not laser diodes), and Ultrasoundepilation.

When it comes to choosing one method over the others, you'll have to take into consideration are budget, tolerance for pain and the health condition of the subject. Permanent hair removal by thermolysis can be done at home, thanks to special devices like The Vector™ Electrolysis Hair Removal System, which are safe and easy to use, and which can get you rid of unwanted hair permanently at a reasonable price.

In electrolysis, the hair is removed directly from the hair follicle. At the same time, the follicle gets destroyed in the process so that the hair won't re-grow. A probe is used and inserted individually into each and every follicle. The probe uses electrical current to cause a chemical reaction which will then loosen the hair so it can be removed. In fact, it causes a chemical reaction that will change the follicle to lye, a product that will damage it enough to cause permanent hair removal. Since each follicle has to be dealt with individually, the process is very time-consuming and thus expensive. Sometimes more than one treatment.

Thermolysis is similar to electrolysis, but heat is used. The process creates heat which destroys the follicle thereby stopping the growth of hair. In addition to pain which is inherent in electrolysis and thermolysis, scarring and burning can occur if performed badly. Although faster than electrolysis, thermolysis is not as effective.

Growing in popularity is the blend method of hair removal. Here, the hair is removed through a quick version of electrolysis where heat is also applied. Lye is produced which destroys the follicle, the heat making it happen faster. The blend method is faster, but still costly and painful.

Despite the various hair removal techniques available, there is still a need for effective, long lasting epilation techniques, and the present invention addresses this need.

### BRIEF SUMMARY OF THE INVENTION

The present invention is directed to providing a method of removing at least one hair from a mammal comprising the steps of (i) associating magnetic field responsive species to the root of at least one hair and (ii) exposing the magnetic responsive species to an oscillating electromagnetic field.

Optionally the magnetic responsive species is selected from the group of materials that reacts with electromagnetic induction.

Typically, the magnetic responsive species is selected from the group consisting of ferromagnetic, ferrimagnetic, conductive and ionized materials.

More typically, the magnetic responsive species is a ferromagnetic species.

Most typically, the magnetic responsive species is a ferrous material.

Optionally the magnetic responsive species are provided to the mammal as a medicinal preparation.

Typically the medicinal preparation is applied to surface of epidermis in vicinity of the hair follicle, and is absorbed into epidermis.

In preferred embodiments, the medicinal preparation is selected from the group comprising ointments, creams, shampoos and lotions.

Alternatively, the medicinal preparation is taken orally.

Alternatively, the medicinal preparation is applied transdermally by injection.

Typically, excess medication is rinsed away from the skin surface.

Optionally, the magnetic responsive species comprises nanoparticles.

Optionally the magnetic responsive is encapsulated within a liposome.

The inductive heating typically uses an oscillating current of frequency between KHz and tens of MHz.

The method may comprise a preliminary step of depilation, which is optionally selected from the group comprising shaving with a razor, shaving with an electric razor and use of a depilatory cream.

Additionally or alternatively, the method comprises a subsequent step of epilation.

The epilation step may comprise waxing, such as by applying melted wax, applying paper or fabric to the melted wax, allowing to harden, and removing the paper or fabric in a smooth motion, thereby removing the hairs therewith.

Buffing may be used to remove weakened hair.

### DESCRIPTION OF THE FIGURES

For a better understanding of the invention and to show how it may be carried into effect, reference will now be made, purely by way of example, to the accompanying Figures, wherewith it is stressed that the particulars shown are by way of example and for purposes of illustrative discussion of the preferred embodiments of the present invention only, and are presented in the cause of providing what is believed to be the most useful and readily understood description of the principles and conceptual aspects of the invention.

FIG. 1 is a schematic illustration of the equipment required for the treatment, and

FIG. 2 is a flow diagram of the steps of a method in accordance with a general embodiment, wherein dashed lines indicate optional steps and solid lines indicate necessary steps.

### DESCRIPTION OF PREFERRED EMBODIMENTS

The present invention is related to epilation by targeting electromagnetic field responsive species to the hair follicles and then applying a magnetic field by induction which causes the species to vibrate and damages the hair follicle or the hair therein by inductive heating and perhaps by physical vibration as well. This causes the hair roots to be damaged and makes the hair fall out, or at least makes hair removal by subsequent waxing a less painful experience.

By magnetic field responsive species, materials that reacts with electromagnetic induction are intended. Typically, the magnetic responsive species is selected from the group consisting of ferromagnetic, ferrimagnetic, conductive and ionized materials. More typically, the magnetic responsive species is a ferromagnetic species. Most typically, the magnetic responsive species is a ferrous material.

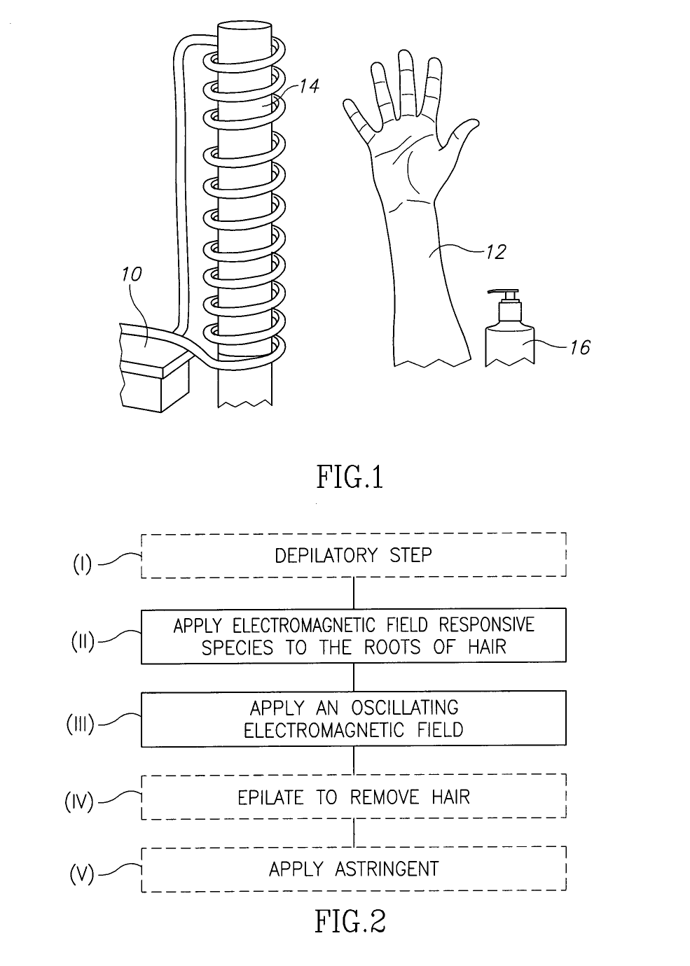

With reference to FIG. 1, the basic setup is an AC power supply 10 that provides electricity with low voltage but very high current and high frequency. The body part 12 to be treated is placed inside an air coil 14 driven by the power supply 10. The alternating magnetic field induced by the AC power supply induces eddy currents in the magnetic particles which heat up and oscillate, thereby damaging the hair follicles.

The process may, by damaging the hair follicles, retard growth of healthy hair, sometimes preventing regrowth completely, and sometimes resulting in brittle, weak hair growth that is easily removed.

There are a number of techniques for directing ferromagnetic particles to the hair follicles.

For example, liposomes and nano particles may be applied as creams or ointments, and preferentially penetrate hair follicles. Both have been considered for drug delivery purposes, though not, to our knowledge, for epilation.

As with laser epilation techniques wherein it may be preferable to first shave the area and then to irradiate the bared skin, preliminary shaving may aid the penetration of the ferromagnetic particles, at least preventing their adhesion to external hair. Alternatively, it may be worth applying an emollient that includes ferromagnetic particles, leaving for sufficient time for the ferromagnetic particles to enter the follicles and then washing or wiping off the superfluous cream, followed by application of an inductive field to damage the hair at the roots. Subsequently, it may be advantageous to apply a conventional waxing or sugaring treatment to remove the hair. In this manner, the hair may be removed less painfully than with conventional waxing or sugaring alone, since the hair roots are damaged. Additionally, the mean period between waxing treatments will be increased.

In some embodiments, an astringent such as witch hazel or aloe vera is applied to dull the associated pain.

A volatile liquid such as alcohol may be applied as a cooling post treatment.

Thus with reference to FIG. 2, in general, methods of the invention consist of the following steps:
(i) an optional depilatory step;
(ii) application of ferromagnetic species to the roots of hair in hair follicles;
(iii) application of an oscillating electromagnetic field to cause inductive heating to the hair follicle
(iv) optional post treatment by epilation to remove hair
(v) optional application of astringent or volatile coolant to ease pain.

### Depilatory Step (Optional)

May use a razor, an electric shaver or depilatory cream

### Application of Ferromagnetic Species to the Roots of Hair in Hair Follicles

Typically the medicinal preparation is applied to surface of epidermis in vicinity of the hair follicle, as an ointment, cream, emollient, shampoo or lotion. Alternatively, the medicinal preparation is taken orally or applied transdermally by injection.

### Application of an Oscillating Electromagnetic Field

This causes inductive heating to the hair follicle. The system of FIG. 1 is used. The strength of the field required depends on the mass of the ferromagnetic particle, its ferromagnetic characteristics, the type of hair to be removed, i.e. pubic, beard, scalp, body hair, the ethnicity of the patient, with some hairs being tougher or more rooted than others, etc.

### Post Treatment by Epilation to Remove Hair (Optional)

This may comprise waxing, sugaring, epilation by plucking, tweezing, threading, use of an epilating machine, such as the Epilady®, etc.

### Application of Astringent or Volatile Coolant (Optional)

It will be appreciated that inductive methods of hair removal, like other permanent treatments such as conventional thermolysis, are painful. Application of astringents, such as Witch Hazel or aloe vera extracts, application of alcohol or other volatiles, and similar post treatments, are advantageous.

The inductive heating may be combined with other methods of hair removal, such as by illumination with strong light, epilation or depilation, etc.

It will be understood that the various embodiments described hereinabove are discussed in minimum detail, and dimensions have not been given. Furthermore, Features shown or discussed with one embodiment may be combined with features described in other embodiments. Thus the scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1. A method of removing at least one hair from a mammal comprising the steps of (i) associating electromagnetic responsive species to the root of at least one hair and (ii) exposing the electromagnetic responsive species to an oscillating electromagnetic field.

2. The method of claim 1, wherein the electromagnetic responsive species is selected from the group of materials that interacts with electromagnetic induction.

3. The method of claim 1 wherein the electromagnetic responsive species is selected from the group consisting of ferromagnetic, ferrimagnetic, conductive and ionized materials.

4. The method of claim 1, wherein the electromagnetic responsive species is a ferromagnetic species.

5. The method of claim 1, wherein the electromagnetic responsive species is a ferrous material.

6. The method of claim 1 wherein the electromagnetic species are provided to the mammal as a medicinal preparation

7. The method of claim 6 wherein the medicinal preparation is applied to surface of epidermis in vicinity of the hair follicle, and is absorbed into epidermis.

8. The method of claim 7, wherein the medication is selected from the group comprising ointments, creams, shampoos and lotions.

9. The method of claim 8 wherein excess medication is rinsed away from the skin surface.

10. The method of claim 1 wherein the medicinal preparation is taken orally.

11. The method of claim 1, wherein the medicinal preparation is applied transdermally by injection.

12. The method of claim 6 wherein the electromagnetic species comprises nanoparticles.

13. The method of claim 6 wherein the electromagnetic species is encapsulated within a liposome.

14. The method of claim 1 comprising a preliminary step of depilation.

15. The method of claim 10 wherein the preliminary step of depilation is selected from shaving with a razor, shaving with an electric razor and use of a depilatory cream.

16. The method of claim 1 comprising a subsequent step of epilation.

17. The method of claim 16 wherein the subsequent step comprises waxing.

18. The method of claim 16 comprises applying melted wax, applying paper or fabric to the melted wax, allowing to harden, and removing the paper or fabric in a smooth motion, thereby removing the hairs therewith.

19. The method of claim 1 comprising inductive heating using an oscillating current of frequency between KHz and tens of MHz.

20. The method of claims 1 comprising applying an astringent.

21. The method of claim 1 wherein the astringent is selected from the group comprising witch hazel and aloe vera.

22. The method of claim 1 comprising applying a volatile liquid as a cooling post treatment.

23. The method of claim 1 followed by a step of buffing to remove weakened hair.

24. (canceled)
